(19) United States
(12) Patent Application Publication
(10) Pub. No.: US 2025/0161899 A1
(43) Pub. Date: May 22, 2025

(54) METHOD FOR MANUFACTURING BUBBLES HAVING A POLYMERIC SHELL USING SOUND WAVES FOR GENERATING THE BUBBLES

(71) Applicants: INSERM (INSTITUT NATIONAL DE LA SANTÉ ET DE LA RECHERCHE MÉDICALE), Paris (FR); UNIV PARIS XIII PARIS-NORD VILLETANEUSE, Villetaneuse (FR); UNIVERSITE PARIS CITE, Paris (FR)

(72) Inventors: Louise FOURNIER, Paris (FR); Cédric CHAUVIERRE, Paris (FR)

(21) Appl. No.: 18/839,574

(22) PCT Filed: Feb. 21, 2022
(86) PCT No.: PCT/IB2022/000095

§ 371 Date: Aug. 19, 2024

(51) Int. Cl. B01J 13/16; A61K 49/22; A61N 7/00

(52) U.S. Cl. CPC B01J 13/16; A61K 49/223; A61N 7/00; A61N 2007/0039

(57) ABSTRACT

The invention relates to an improved method for the manufacturing of bubbles having a polycyanoacrylate shell using sound waves for generating the bubbles. It has been found that the application of sound waves for at least 6 minutes in combination with the choice of a specific pH was critical to produce stable bubbles with a high yield. The bubbles produced by the method of the invention have an improved sphericity compared to the bubbles obtained with the methods of the prior art and are obtained in shorter times. With the method of the invention, the bubbles can be functionalized with fluorescent probes or with polysaccharides, which can confer targeting properties to the bubbles. Such bubbles can be used as contrast agents for ultrasound imaging or for sonothrombolysis.

The present invention relates to a method for manufacturing bubbles having a polymeric shell and more particularly to a method wherein the bubbles are generated by applying sound waves to an aqueous solution and wherein the polymeric shell is formed by polymerization of a cyanoacrylate monomer.

Micro- and nanobubbles are nowadays used in various biomedical applications. While the most common application of such bubbles is as contrast agents for ultrasound imaging, they have also been used as delivery vehicles (e.g. for drugs, genes or for O₂ delivery) and as thrombolytic agents.

Commercially available bubbles include those sold under the brand names Sonazoid™ and Optison™ (produced by GE Healthcare), Sonovue™ (produced by Bracco) and Definity™ (produced by Lantheus Medical Imaging).

Micro- and nanobubbles generally comprise a gaseous core and a stabilizing shell. Said shell is generally made of surfactants, lipids or proteins. Bubbles having a polymeric shell have also been studied as they can be more stable and can attract less immune response in vivo than bubbles having a proteinic shell.

Bubbles having a polymeric shell made of polycyanoacrylate have been described in Bo Li et al., Functionalized polymer microbubbles as new molecular ultrasound contrast agent to target P-selectin in thrombus, Biomaterials, 2019, Volume 194, Pages 139-150. The bubbles having a polymeric shell made of polycyanoacrylate described therein are obtained by hydrodynamic cavitation. However, the reaction time is of 60 minutes and the bubbles obtained are not homogeneous in shape and generally not spherical.

It would thus be advantageous to be able to produce bubbles having a polymeric shell made of polycyanoacrylate requiring shorter reaction times and being more homogeneous in shape and more spherical.

The present invention discloses a method for manufacturing bubbles having a polymeric shell made of polycyanoacrylate using sound waves to generate the bubbles instead of hydrodynamic cavitation. Surprisingly, the method of the invention allows to obtain bubbles having a polymeric shell made of polycyanoacrylate with reduced reaction times compared to the method using hydrodynamic cavitation described in Bo Li et al.

It has been found that the application of sound waves for at least 6 minutes in combination with the choice of a specific pH was critical to produce with a high yield stable bubbles having a polymeric shell. This is surprising since it could have been expected that such sonication times would have degraded the structures formed. Indeed, acoustic cavitation is known to generate locally elevated temperature and free radicals that can degrade polymeric molecules.

Moreover, the bubbles obtained with the method of the present invention unexpectedly have an improved sphericity than the bubbles obtained using hydrodynamic cavitation.

### SUMMARY OF THE INVENTION

One aspect of the present invention is related to a method for manufacturing bubbles having a polymeric shell comprising the following steps:
a) providing a first volume of an aqueous solution comprising a gas dissolved therein, a surfactant dissolved therein, and having a pH below 6, preferably comprised between 1.5 and 5, more preferably comprised between 2 and 4;
b) in the aqueous solution, generating bubbles comprising the gas by applying sound waves to the aqueous solution;
c) while generating the bubbles, adding a second volume of a cyanoacrylate monomer to the aqueous solution, thereby forming a suspension of the bubbles having a polymeric shell in the aqueous solution, wherein said polymeric shell is formed by polymerization of the cyanoacrylate monomer at the interface between the aqueous solution and the bubbles;
d) recovering the bubbles having a polymeric shell;
   characterized in that the sound waves in step b) are applied during a period of at least 6 minutes, preferably between 8 and 30 minutes, more preferably between 15 and 30 minutes after the beginning of the addition of the cyanoacrylate monomer.

Another aspect of the present invention is related to the bubbles obtainable by the method according to the invention.

### DESCRIPTION OF THE FIGURES

FIG. 1 compares the shape and the ability to incorporate a fluorescent probe of:
Top line: bubbles obtained according to the prior art Bo Li et al., Functionalized polymer microbubbles as new molecular ultrasound contrast agent to target P-selectin in thrombus, Biomaterials, 2019, Volume 194, Pages 139-150;
Middle line: bubbles comprising Nile red as a fluorescent probe obtained by the method of Example 5;
Bottom line: bubbles comprising Nile red as a fluorescent probe obtained by the method of Example 4.

In each line, the left image shows the fluorescence of Nile red in the sample, the middle image shows the same view without fluorescence and the right image shows the left image and the middle image merged.

On the left and right images, the lighter parts correspond to fluorescence.

The scale bar on the images corresponds to 10 μm.

It can be seen that the bubbles obtained by the method of Example 5 (middle line), wherein the amplitude of vibration of the tip is set at 369 μm and the sound waves are applied for 10 minutes, did not incorporate fluorescence as efficiently as the bubbles obtained by the method of Example 4 (bottom line), wherein the vibration of the tip is set at 243 μm and the sound waves are applied for 10 minutes. Moreover, the bubbles obtained by the method of the invention (middle and bottom lines) are more spherical and homogeneous in shape than the bubbles obtained according to the prior art (top line).

FIG. 2 shows a scanning electron microscopy image of the bubbles obtained using the method described in Example 1. The scale bar on the image corresponds to 4 μm.

### DETAILED DESCRIPTION OF THE INVENTION

An object of the present invention is a method for manufacturing bubbles having a polymeric shell comprising the following steps:
a) providing a first volume of an aqueous solution comprising a gas dissolved therein, a surfactant dissolved therein, and having a pH below 6, preferably comprised between 1.5 and 5, more preferably comprised between 2 and 4;
b) in the aqueous solution, generating bubbles comprising the gas by applying sound waves to the aqueous solution;
c) while generating the bubbles, adding a second volume of a cyanoacrylate monomer to the aqueous solution, thereby forming a suspension of the bubbles having a polymeric shell in the aqueous solution, wherein said polymeric shell is formed by polymerization of the cyanoacrylate monomer at the interface between the aqueous solution and the bubbles;
d) recovering the bubbles having a polymeric shell;
   characterized in that the sound waves in step b) are applied during a period of at least 6 minutes, preferably between 8 and 30 minutes, more preferably between 15 and 30 minutes after the beginning of the addition of the cyanoacrylate monomer.

In step b) of the method of the invention, the bubbles can be generated directly by the sound waves when they lower the pressure of the aqueous solution enough to produce bubbles. In step b), the bubbles can also be generated by the fragmentation or coalescence of other bubbles, said fragmentation or coalescence being itself caused or accelerated by the sound waves. Said other bubbles can themselves be generated directly by the sound waves and/or can originate from e.g. gas bubbling through the aqueous solution, and/or gas trapped at the interface between the aqueous solution and any instrument plunged therein (e.g. a sonotrode), and/or gas trapped at the interface between the aqueous solution and the walls of the reactor.

The bubbles generated by the sound waves in step b) contain the gas dissolved in the aqueous solution together with water vapor at least because of the partition equilibrium between the bubbles and the aqueous solution.

In the method of the invention, the bubbles having a polymeric shell recovered at step d) originate from the bubbles generated by the sound waves in step b). Because of the partition equilibrium between the bubbles and the aqueous solution, the bubbles having a polymeric shell recovered at step d) can be enriched in the gas dissolved in the aqueous solution compared to the bubbles generated by the sound waves in step b).

The gas dissolved in the solution can be one gaseous compound or a mixture of gaseous compounds or a mixture of compounds different from water that would be gaseous in the conditions of temperature and pressure used when the method of the invention is performed.

Preferably, in the method according to the invention, the gas dissolved in the solution has a molar mass of at least 100 g/mol, more preferably of at least 200 g/mol.

Preferably, in the method according to the invention, the gas dissolved in the solution has a density at 20° C. and 1013 mbar of at least 7 kg/m³, more preferably at least 9 kg/m³.

Preferably, in the method according to the invention, the gas dissolved in the solution is selected from the group consisting of alkanes, fluoroalkanes, and mixtures thereof, more preferably the gas dissolved in the solution is a perfluoroalkane, such as perfluoropropane or perfluorobutane, or a mixture of perfluoroalkanes, even more preferably the gas dissolved in the solution is perfluorobutane.

Surprisingly, the bubbles are obtained in particularly high yields when the gas dissolved in the solution is selected from the group consisting of alkanes, fluoroalkanes, and mixtures thereof, in particular when the gas dissolved in the solution is a perfluoroalkane, such as perfluoropropane or perfluorobutane, or a mixture of perfluoroalkanes, more particularly when the gas dissolved in the solution is perfluorobutane.

Preferably, in the method according to the invention, the amount of the gas dissolved in the aqueous solution is maintained over 0.5 mg/L throughout steps b) and c), more preferably the aqueous solution is saturated with said gas throughout steps b) and c).

A gas can be dissolved in an aqueous solution by bubbling said gas through the aqueous solution.

Preferably, in the method according to the invention, the gas dissolved in the aqueous solution is bubbled through the aqueous solution during steps b) and c), preferably gas dissolved in the aqueous solution is bubbled through the aqueous solution throughout steps b) and c).

The surfactant contributes to the formation of the bubbles and to their stabilization.

Preferably, in the method according to the invention, the surfactant is a nonionic surfactant, for example a polysorbate, a poloxamer, an octylphenol (including ethoxylated derivatives of octylphenols), a nonylphenol (including ethoxylated derivatives of nonylphenols), an ethoxylated fatty alcohol (e.g. a cetomacrogol), a macrogol-glycerol ester (e.g. an ethoxylated hydrogenated castor oil), or a mixture thereof.

Preferably, in the method according to the invention, the surfactant has a hydrophilic-lipophilic balance comprised between 8 and 18, more preferably comprised between 10 and 18, even more preferably comprised between 12 and 18.

Preferably, in the method according to the invention, the amount of surfactant in the first volume of the aqueous solution of step a) is of at least 10 times the critical micelle concentration (CMC) of said surfactant, more preferably of at least 50 times the CMC of said surfactant.

Preferably, in the method of the invention, a source of the sound waves is plunged in the aqueous solution.

Sound waves can be applied in a pulsed mode. As defined herein in a pulsed mode, the sound waves are not applied continuously for more than 10 seconds.

Preferably, in the method according to the invention, the sound waves are not applied in a pulsed mode, more preferably the time interval between two periods of application of the sound waves is lower than 10 seconds, even more preferably the sound waves are applied continuously in one time.

Preferably, in the method according to the invention, the source of the sound waves vibrates with an amplitude comprised between 50 μm and 500 μm, more preferably between 100 μm and 500 μm, even more preferably between 200 μm and 400 μm, more particularly the amplitude of the sound waves is comprised between 200 μm and 300 μm.

Increasing the amplitude of vibration of the source of the sound waves generally accelerates the formation of the microbubbles having a polymeric shell. It also results in a faster increase of the temperature of the aqueous solution.

In a particular embodiment of the method of the invention, the source of the sound waves vibrates with an amplitude comprised between 200 μm and 400 μm and the period of application of the sound waves after the beginning of the addition of the cyanoacrylate monomer is of between 8 and 30 minutes.

In a particular embodiment of the method of the invention, the source of the sound waves vibrates with an amplitude comprised between 200 μm and 300 μm and the period of application of the sound waves after the beginning of the addition of the cyanoacrylate monomer is of between 15 and 30 minutes.

Surprisingly, with the above conditions of amplitude of vibration for the source of the sound waves and of duration of the application of the sound waves, better incorporation of fluorescent probes is observed, as shown in FIG. 1.

Preferably, in the method according to the invention, the frequency of the sound waves is comprised between 10 kHz and 100 kHz, more preferably between 15 kHz and 50 kHz.

Preferably, in the method according to the invention, an emitting surface of the source of the sound waves plunged in the aqueous solution is of at least 1 mm² per 10 mL of the aqueous solution.

Preferably, in the method according to the invention, the second volume of the hydrophobic cyanoacrylate monomer corresponds to at least 0.01 vol. %, preferably between 0.5 vol. % and 5 vol. %, of the first volume of the aqueous solution.

Preferably, in the method according to the invention, the addition of the second volume of the hydrophobic cyanoacrylate monomer begins less than 5 minutes, more preferably less than 1 minute, even more preferably between 10 seconds and 1 minute after the beginning of step b).

Preferably, in the method according to the invention, the cyanoacrylate monomer is an alkyl cyanoacrylate monomer, more preferably wherein said alkyl group comprises from 2 or 8 carbon atoms, even more preferably wherein said alkyl group is a n-butyl or isobutyl group.

It is believed that the cyanoacrylate monomer has some affinity for the interface between the bubbles and the aqueous solution, allowing the formation of the polymeric shell when the cyanoacrylate monomer polymerizes.

Cyanoacrylate monomer can polymerize through anionic polymerization. In water, higher pH are more favorable to anionic polymerization of cyanoacrylate monomers than lower pH. In the method according to the invention, the pH of the first volume of the aqueous solution is chosen to prevent the anionic polymerization of the cyanoacrylate monomer to be too fast or too slow. The pH of the first volume of the aqueous solution is thus chosen so that the cyanoacrylate monomer polymerizes slow enough for it to have time to migrate to the interface between the bubbles and the aqueous solution and at the same time polymerizes fast enough for the polymeric shell to be formed before the bubbles are destabilized.

In step c), the cyanoacrylate monomer can be added at a controlled speed or all at once. Preferably the time of addition of the cyanoacrylate monomer does not exceed 5 minutes and is such that the sound waves are applied after the end of the addition of the cyanoacrylate monomer for at least 4 minutes.

In some embodiments of the method according to the invention, the first volume of aqueous solution of step a) further comprises a saccharide or a derivative thereof, for example an oligosaccharide, a polysaccharide, or a derivative thereof, so as to produce bubbles having a polymeric shell wherein the saccharide or derivative thereof is copolymerized with the cyanoacrylate monomer.

Preferably, said saccharide or derivative thereof is present in the first volume of aqueous solution of step a) in an amount of 0.1 to 4% by weight relative to the weight of the first volume of aqueous solution.

It is believed that the saccharides or derivatives thereof copolymerize with the cyanoacrylate via addition of the hydroxyl groups in the saccharides or derivatives thereof to the vinylic group of the cyanoacrylate monomers.

Preferably, in the method according to the invention, the saccharide or derivative thereof is selected from the group consisting of fucoidans, dextrans, mannans, derivatives of fucoidans, derivatives of dextrans (such as carboxymethyl dextran, carboxymethyl dextran FITC, dextran FITC), derivatives of mannans (such as glucomannan and galactomannan), and mixtures thereof.

Including a saccharide in the shell of the bubbles potentially gives them further functionalities. For example, bubbles functionalized with fucoidan have been shown to efficiently target the protein P-selectin, bubbles functionalized with a fluorescent saccharide such as dextran FITC have fluorescent properties useful for optical imaging purposes.

In some embodiments of the method according to the invention, the first volume of aqueous solution of step a) and/or the second volume of the cyanoacrylate monomer added in step c) further comprises a fluorescent probe so as to yield polymer-coated bubbles comprising the fluorescent probe, preferably said fluorescent probe is present in an amount of 0.1 to 40 ppm by weight relative to the weight of the first volume of aqueous solution.

In some embodiments of the method according to the invention, a fluorescent probe is incorporated in the bubbles having a polymeric shell, after steps b) and c) or at any later stage in the method, by adding said fluorescent probe to a suspension of the bubbles having a polymeric shell.

Surprisingly, it has been observed that these embodiments of the method allow to incorporate the fluorescent probe in the bubbles having a polymeric shell in such a way that the fluorescent probe is not released upon subsequent washing of the bubbles.

Preferably, when the fluorescent probe is added to the first volume of aqueous solution of step a) or after steps b) and c), said fluorescent probe is soluble in water, for example said fluorescent probe is rhodamine B.

Preferably, when the fluorescent probe is added to the second volume of the cyanoacrylate monomer added in step c), said fluorescent probe is lipophilic, for example said fluorescent probe is Nile red.

Preferably, in the method according to the invention, steps b) and c) are performed while maintaining the temperature of the aqueous solution below 60° C., more preferably below 35° C.

Applying the sound waves to the aqueous solution generally results in an increase of the temperature of the aqueous solution during step b) and c) of the method of the invention. It is preferred to prevent excessive heating of the aqueous solution. It has thus been observed that when the temperature of the aqueous solution reaches 70° C. at the end of step b) lower yields in number of bubbles per mL are obtained. Maintaining the temperature of the aqueous solution below 60° C., more preferably below 35° C. can be performed by any means known of the person skilled in the art, e.g. by using an ice bath.

In addition to maintaining the temperature of the aqueous solution below 60° C., more preferably below 35° C., the first volume of aqueous solution just before step b) preferably has a temperature below 10° C.

Surprisingly, in the embodiments of the method of the invention where an oligosaccharide, a polysaccharide, or a derivative thereof, able to target a protein is comprised in the first volume of aqueous solution of step a), it has been observed that the bubbles having a polymeric shell obtained had improved targeting properties when the temperature of the first volume of aqueous solution just before step b) was below 10° C.

Preferably, in the method according to the invention, the first volume of the aqueous solution is obtained by the following steps:
a1. acidifying a volume a0 of an aqueous solution with a strong inorganic acid, such as HCl, H₂SO₄, or HNO₃, to obtain the first volume of the aqueous solution having a desired pH;
a2. dissolving the gas in the first volume of the aqueous solution to obtain the first volume of the aqueous solution comprising the gas dissolved therein and having the desired pH;
a3. Optionally, cooling the first volume of the aqueous solution comprising the gas dissolved therein and having the desired pH, so as to reach a temperature below 10° C.;
a4. While preventing of the aqueous solution from becoming depleted in the hydrophobic gas, preferably by bubbling the gas through the aqueous solution, and, optionally, while maintaining the aqueous solution at a temperature below 10° C., adding the surfactant to obtain the first volume of the aqueous solution comprising the gas dissolved therein, the surfactant dissolved therein, and having the desired pH.

A desired pH is below 6, in particular comprised between 1.5 and 5, more particularly comprised between 2 and 4.

Preferably, in the method according to the invention, step b) starts less than 30 s after the surfactant has been added.

Surprisingly, in the embodiments of the method of the invention where an oligosaccharide, a polysaccharide, or a derivative thereof, is comprised in the first volume of aqueous solution of step a), it has been observed that the bubbles having a polymeric shell obtained showed better incorporation of the oligosaccharide, polysaccharide, or derivative thereof, when step b) starts less than 30 s after the surfactant has been added.

Preferably, in the method according to the invention, the saccharide is comprised in the aqueous solution before step a3, more preferably the saccharide is added to the aqueous solution before step a2, either before or after the acidification of step a1.

Preferably, in the method according to the invention, step d) comprises a step d1) of centrifugating the aqueous solution obtained after steps b) and c) and separating the bubbles having a polymeric shell from the rest of the aqueous solution, for example, said centrifugation is performed at a relative centrifugal force (RCF) comprised between 0.01 and 1, more preferably between 0.05 and 0.2.

Preferably, in the method according to the invention, step d) comprises, after step d1), a step d2) of washing the separated bubbles having a polymeric shell on a sieve, more preferably using a sieve shaker, to obtain a suspension of the polymer-coated bubbles with a size smaller than the sieve opening, preferably the sieve opening is lower than 10 μm, more preferably lower than 5 μm.

Preferably, in the method according to the invention, the washing of step d2) is performed using a washing solution comprising water and less than 0.1%, preferably less than 0.05% by mass of a surfactant. In a preferred embodiment, said surfactant is the same as the surfactant comprised in the first volume of aqueous solution.

Preferably, in the method according to the invention, step d) comprises, after step d1) and, where applicable before step d2), a step d1bis) of resuspending the separated bubbles having a polymeric shell in the washing solution and reseparating the bubbles having a polymeric shell through centrifugation, for example, the centrifugation of step d1bis) is performed at a relative centrifugal force (RCF) comprised between 0.01 and 1, preferably between 0.05 and 0.2.

Preferably, in the method according to the invention, step d1bis) is performed at least two times.

Preferably, in the method according to the invention, step d) comprises, after the last one of steps d1), d1bis), and d2), where applicable, a step d3) of suspending the bubbles having a polymeric shell in a storage solution, more preferably the storage solution is identical to the washing solution.

Preferably, in the method according to the invention, step d) comprises, after the last one of steps d1), d1bis), d2) and d3), where applicable, a step d4) of storing the suspension of the bubbles having a polymeric shell at a temperature of less than 10° C.

When stored in suspension in the storage solution at a temperature of less than 10° C., the bubbles obtained by the method of the invention are stable at least 6 weeks, as evidenced from size measurements.

Preferably, in the method according to the invention, the obtained bubbles having a polymeric shell have a smallest dimension of less than 10 μm, more preferably less than 7 μm, even more preferably less than 5 μm.

For example, bubbles having a polymeric shell having a smallest dimension of less than 10 μm can be obtained by using a sieve having a sieve opening of less than 10 μm, for example as in step d2).

For biomedical applications, it is generally required that the smallest dimension of the bubbles remains below 10 μm so that they do not block blood flow when they are administered intravenously.

Preferably, in the method according to the invention, the obtained bubbles having a polymeric shell have a volume-weighted mean diameter (Dv(50)) below 4 μm, for example comprised between 1 μm and 4 μm, as measured by laser light scattering using the Mie model assuming that the solvent is water with a refraction index of 1.330 and that the bubbles are spherical polystyrene latex particles with a refraction index of 1.590 and an extinction coefficient (imaginary part of the complex refractive index) of 0.010.

Another object of the present invention are bubbles obtainable by the method of the invention.

Preferably the bubbles obtainable by the method of the invention are spherical.

In some embodiments, the bubbles obtainable by the method of the invention can be used in a method of treatment and/or of diagnosis. For example, they can be used as a contrast agent in a method of diagnosis. In another example, they can be used in sonothrombolysis.

### EXAMPLES

### Bubble Size Measurements

The size of the bubbles is measured thanks to a laser diffraction particle size analyzer (Mastersizer 3000, Malvern Instruments).

For analysis, the Mie model is used assuming that the solvent is water with a refraction index of 1.330 and that the bubbles are spherical polystyrene latex particles with a refraction index of 1.590 and an extinction coefficient (imaginary part of the complex refractive index) of 0.010. The analysis yields values for the surface-weighted mean diameter (D[3,2]), the volume weighted mean diameter (D[4,3]), the Dv(10), the Dv(50) and the Dv(90) (the point in the size distribution, up to and including which, 10%, respectively 50%, resp. 90% of the total volume of material in the sample is contained).

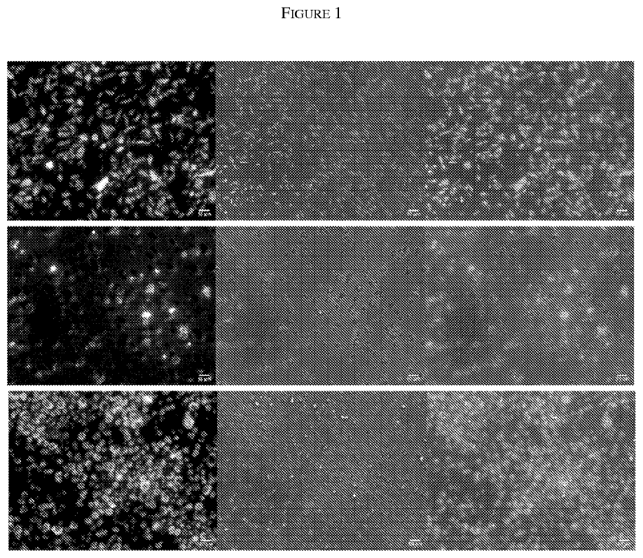

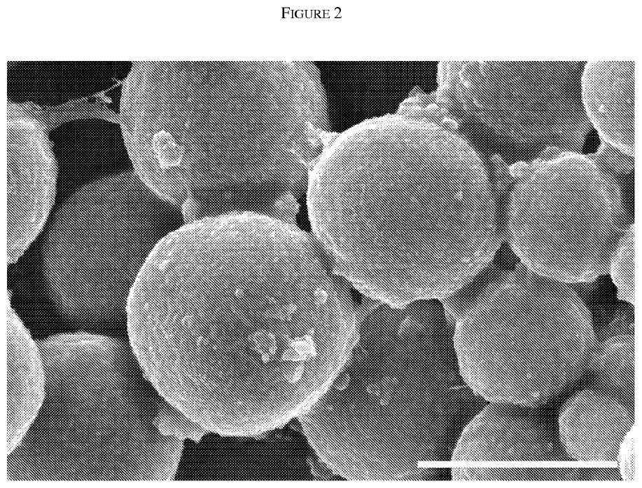

Although these values for the optical properties of the bubbles are not accurate, the result of the analysis is still useful for comparison purposes. For example, the measurements can be reproduced over 6 weeks which demonstrate the shelf-stability of the bubbles. Moreover, the value obtained for the median particle size by volume (Dv(50)) is close to the diameter observed by scanning electron microscopy or by optical microscopy (see FIGS. 1 and 2).

### Bubble Concentration Measurement

The bubble concentration is expressed in number of bubbles per mL and is measured with image analysis. Briefly, a diluted solution of bubbles is injected into a hemocytometer (counting chamber device for cell counting) and pictures are taken with an optical microscope (Zeiss). Bubbles are isolated thanks to ImageJ thresholding and three small squares are averaged for each sample allowing an evaluation of the mean concentration with count extrapolation.

### Example 1: Synthesis of Bubbles Having a Poly(Isobutyl Cyanoacrylate) (PIBCA) Shell According to the Invention

50 mL of an aqueous solution are prepared with water and HCl 1M to reach a pH of 2.5. 500 μL of Tween 20 are added to the aqueous solution. A perfluorobutane (PFB) tank is connected to the aqueous solution with a polyethylene catheter (Bioseb Lab Instruments) and PFB is bubbled through the solution. Meanwhile, 700 μL of isobutyl cyanoacrylate (IBCA) are loaded into a 1 mL syringe and placed onto a syringe pump (AL-300, World Precision Instrument, Florida, USA). The aqueous solution is then placed in ice, under the sonotrode (model 101-148-070, Branson Ultrasonic™; diameter of the tip: 3 mm) of a sonicator (Sonifier™ SFX 550, Branson Ultrasonic™) with the tip of the sonotrode centered relative to the aqueous solution and with half of a centimeter of the tip plunged into the aqueous solution. The IBCA is connected to the aqueous solution with a polyethylene catheter (Bioseb Lab Instruments). The amplitude of vibration of the tip is set at 243 μm. The sonicator is turned on in continuous mode, producing sound waves with a frequency of 20 kHz for 20 min, and the IBCA is injected at a controlled speed of 0.45 mL/min about 30 seconds after the sonication begins.

The obtained foam is centrifuged 3 times at 0.1 relative centrifugal force (RCF) in 50 mL Falcon™ tubes for 20 min, and a solution of 0.02% Tween 20 is used to resuspend the floating bubbles. The bubbles are then washed through a 5 μm sieve using a sieve shaker. The obtained suspension is stored at 5° C. in a 0.02% Tween 20 solution.

### Example 2: Synthesis of Bubbles Having a PIBCA Shell According to the Invention

The same procedure as example 1 is followed except that the amplitude of vibration of the tip is set at 369 μm and that the sound waves are applied for 10 min.

### Example 3: Synthesis of Bubbles Having a PIBCA Shell Functionalized with Polysaccharides According to the Invention

The same procedure as example 1 is followed except that 500 mg (1%) to 1 g (2%) of the polysaccharide are dissolved in the aqueous solution prior to adjusting its pH to 2.5, that the solution is put into a freezer for 15 min to cool its temperature down to 5° C. before adding the Tween 20 and that the Tween 20 is added just before the beginning of the sonication.

### Example 4 and 5: Synthesis of Bubbles Having a PIBCA Shell Functionalized with Nile Red According to the Invention

The same procedures as examples 1 and 2, respectively, are followed except that the IBCA injected contained 30 μg of Nile red, added to the IBCA as a solution in acetone at 1 mg/mL.

### Example 6: Effect of the Duration and Intensity of the Acoustic Cavitation

The same procedure as example 1 is followed except for the duration and amplitude of the sonication, which are as described in Table 1. It can be seen that no bubbles are obtained when the duration of the sonication is of 5 minutes whereas for durations of 10 minutes and 20 minutes, stable bubbles are obtained with a high yield.

TABLE 1

| | Dura- | Ampli- | | Dv | Dv | Dv | |
|---|---|---|---|---|---|---|---|
| Test | tion | tude | Bubbles | (10) | (50) | (90) | |
| No. | (min) | (μm) | per mL | (μm) | (μm) | (μm) | Observations |
| 4a | 5 | 243 | — | — | — | — | no stable |
| | | | | | | | bubble |
| 4b | 5 | 306 | — | — | — | — | no stable |
| | | | | | | | bubble |
| 4c | 5 | 369 | — | — | — | — | no stable |
| | | | | | | | bubble |
| 4d | 5 | 431 | — | — | — | — | no stable |
| | | | | | | | bubble |
| 2 | 10 | 369 | 3.2 · 10¹⁰ | 0.35 | 2.91 | 6.01 | bubbles are |
| | | | | | | | stable |
| 1 | 20 | 243 | 2.5 · 10¹⁰ | 0.37 | 3.27 | 6.40 | bubbles are |
| | | | | | | | stable |

1. Method for manufacturing bubbles having a polymeric shell comprising the following steps:
a) providing a first volume of an aqueous solution comprising a gas dissolved therein, a surfactant dissolved therein, and having a pH below 6;
b) in the aqueous solution, generating bubbles comprising the gas by applying sound waves to the aqueous solution;
c) while generating the bubbles, adding a second volume of a cyanoacrylate monomer to the aqueous solution, thereby forming a suspension of the bubbles having a polymeric shell in the aqueous solution, wherein said polymeric shell is formed by polymerization of the cyanoacrylate monomer at the interface between the aqueous solution and the bubbles;
d) recovering the bubbles having a polymeric shell;
characterized in that the sound waves in step b) are applied during a period of at least 6 minutes after the beginning of the addition of the cyanoacrylate monomer.

2. (canceled)

3. (canceled)

4. The method according to claim 1, wherein the gas dissolved in the solution is selected from the group consisting of alkanes, fluoroalkanes, and mixtures thereof.

5. (canceled)

6. The method according to claim 1, wherein the gas dissolved in the aqueous solution is bubbled through the aqueous solution during steps b) and c).

7. The method according to claim 1, wherein the surfactant is a nonionic surfactant.

8. The method according to claim 1, wherein the surfactant has a hydrophilic-lipophilic balance comprised between 8 and 18.

9. The method according to claim 1, wherein the amount of surfactant in the first volume of the aqueous solution of step a) is of at least 10 times the critical micelle concentration (CMC) of said surfactant.

10. (canceled)

11. (canceled)

12. The method according to claim 1, wherein the source of the sound waves vibrates at an amplitude comprised between 50 μm and 500 μm.

13. The method according to claim 1, wherein the frequency of the sound waves is comprised between 10 kHz and 100 kHz.

14. (canceled)

15. The method according to claim 1, wherein the second volume of the cyanoacrylate monomer corresponds to at least 0.01 vol. %.

16. (canceled)

17. The method according to claim 1, wherein the first volume of aqueous solution of step a) further comprises a saccharide or a derivative thereof so as to produce the bubbles having a polymeric shell wherein the saccharide or derivative thereof is copolymerized with the cyanoacrylate monomer.

18. (canceled)

19. (canceled)

20. (canceled)

21. The method according to claim 1, wherein steps b) and c) are performed while maintaining the temperature of the aqueous solution below 60° C., or below 35° C.

22. The method according to claim 21, wherein the first volume of aqueous solution just before step b) has a temperature below 10° C.

23. (canceled)

24. (canceled)

25. The method according to claim 1, wherein step b) starts less than 30 s after the surfactant has been added.

26. (canceled)

27. (canceled)

28. (canceled)

29. (canceled)

30. (canceled)

31. (canceled)

32. (canceled)

33. The method according to claim 1, wherein the obtained bubbles having a polymeric shell have a smallest dimension of less than 10 μm.

34. The method according to claim 1, wherein the obtained bubbles having a polymeric shell have a median diameter by volume below 4 μm as measured by laser light scattering using the Mie model assuming that the solvent is water with a refraction index of 1.330 and that the bubbles are spherical polystyrene latex particles with a refraction index of 1.590 and an extinction coefficient of 0.010.

35. Bubbles obtainable by the process according to claim 1.

36. Bubbles according to claim 35, said bubbles being spherical.

37. (canceled)

38. (canceled)

39. (canceled)

40. A contrast agent comprising the bubbles according to claim 35.

41. A sonothrombolytic agent comprising the bubbles according to claim 35.
